(19) United States
(12) Patent Application Publication
(10) Pub. No.: US 2006/0212049 A1
(43) Pub. Date: Sep. 21, 2006

(54) Pyeloplasty clip

(76) Inventor: Mohammed M. Mohiuddin, Frederick, MD (US)

Correspondence Address: LAUBSCHER & LAUBSCHER, P.C., 1160 SPA ROAD, SUITE 2B, ANNAPOLIS, MD, 21403, US

(21) Appl. No.: 11/080,473

(22) Filed: Mar. 16, 2005

(51) Int. Cl. A61B 17/08

(52) U.S. Cl. 606/151

(57) ABSTRACT

A pyeloplasty clip for surgical use is operable in a first condition to clamp together portions of human tissue that are to be severed. The clip is then operable in a second condition either to separate and maintain in an open condition the severed tissue portions, or to displace the clamped severed tissue portions toward each other. A fastener arrangement serves to maintain together the clamping leg portions of the clip until the human body has healed. A separating device may be provided for subsequently unfastening and opening the clamping leg portions, thereby to permit removal of the clip from the human body.

### BACKGROUND OF THE INVENTION

1. Field of the Invention

A pyeloplasty clip for surgical use is operable in a first condition to clamp together portions of human tissue that are to be severed or joined. The clip is then operable in a second condition either to separate and maintain in an open condition the severed tissue portions, or to displace the clamped tissue portions toward each other. Fastener means serve to maintain together the clamping leg portions until the human body has healed, and separating means are provided for subsequently unfastening and opening the clamping leg portions.

2. Description of the Related Art

The provision of various types of surgical clips is well known in the patented and published prior art for performing gastroesphageal fund oplication, as evidenced by the published patent applications of Sixto, et al., Nos. 2002/0068945 and 2002/0138086, and Kortenbach, et al., No. 2002/0068946.

In Monassevitch, et al., published application No. 2002/0177859, a surgical grip applicator device includes means for gripping and positioning a surgical clip relative to hollow organ portions, and releasing means for releasing the clip. Tissue cutting apparatus is provided for cutting through the tissue walls.

The Yoon U.S. Pat. No. 5,217,473 relates to multi-function surgical instruments including stretch ligating and occluding devices. The surgical devices include leg portions that may be provided with serrated legs, and with mating pin and aperture engaging means which enhance the locking of the clip onto the tissue being occluded.

The present invention was developed to provide an improved pyeloplasty clip that is operable in a first condition to clamp together portions of human tissue that are to be joined and severed, and in a second condition to maintain the tissue portions in a desired open or closed relation during healing.

### BRIEF SUMMARY OF THE INVENTION

Accordingly, a primary object of the present invention is to provide an improved pyeloplasty clip including a unitary clip body having four adjacent spaced linear leg portions, a first insertion portion connecting adjacent ends of first and second ones of the leg portions, a second insertion portion connecting corresponding adjacent ends of third and fourth leg portions, a first joinder portion connecting the other ends of the first and third leg portions, and a second joinder portion connecting the other ends of the second and fourth leg portions. The clip body normally has a first expanded condition in which the first and second insertion portions and the associated leg portions are spaced to define a chamber for receiving the human tissue that is to be treated. The clip body is operable to a clamped second condition by bending of the joinder portions to displace the insertion portions together into clamping engagement with the human tissue contained therebetween.

According to another object of the invention, the clamped tissue may be severed along a line of severing extending from a point between the joinder portions of the clip toward the insertion portions, whereupon the clip is operable toward a separated third condition in which, as a result of the bending of the first and second insertion portions, the first joinder and associated leg portions are displaced apart from the second joinder and associated leg portions, thereby to maintain the clamped severed human tissue portions in a separated condition. Alternatively, in the case of severing for the removal of human tissue, the first and second joinder portions and associated leg portions could be displaced together toward a closed position for healing of the human body.

Another object of the invention is to provide fastening means operable to fasten together the first and third leg portions and the second and fourth leg portions when the clip is in the clamping second condition. Fastening pins extend from one leg through the clamped tissue into corresponding fastening locking openings contained in the associated leg on the other side of the tissue. Separating means may be provided for separating the fastened leg portions after the human body has healed.

The pyeloplasty clip of the present invention is a very small and simple device for increasing the size of and initial bifurcation between any tow organs, tubes, arteries or veins. One application of the clip is for congenital uretero-pelvic-junction (UPJ) with hydronephrosis. In such cases, the UPJ is usually occluded and the pyeloplasty clip with its associated surgical procedure, is used to enlarge the occlusion and to maintain the opening with an adequate area for the flow of fluids. In this case, the clip is removed after the healing period of about 2 to 3 weeks. In the case of “foreign body” insertions in urinary tracts, the removal of the clip is critical because of possible long-term deposits on the foreign body and stone formation. This simple procedure with the pyeloplasty clip greatly reduces the recuperation time of the individual as compared to the prevalent procedures of endo-pyelotomy. An open procedure normally takes about two hours of operating time in the surgery operating room, but with the use of the clip of the present invention, the procedure requires minimal surgery and can be done on an outpatient basis or with a maximum of one day of hospitalization. The procedure produces minimum suffering, and the reduction of occlusion to fluid flow is comparable to open surgery.

Another application of the pyeloplasty clip of the present invention is the reduction of constricted arterial bifurcations. In these cases, the clip is permanently left inside the artery to maintain the enlarged area at the bifurcation.

The pyeloplasty clip is applicable to all bifurcations that require the lumen to be opened up and expanded, wherever there is a need for enlargement of a bifurcation. The thin clip is inserted from the larger lumen of the connecting organ or tube at the location of the bifurcation. The area of the initial bifurcation is increased either by an added manual incision between the legs of the clip, or in anther design, the incision is automatically achieved when the legs of the clip are joined. In some cases, the clip is left inside the organ, while in other situations, the clip is removed by means of a flexible string arrangement. The clip may be formed from stainless steel, shape memory retaining alloys, or from a synthetic plastic material having similar resilient properties.

### BRIEF DESCRIPTION OF THE DRAWINGS

Other objects and advantages of the invention will become apparent from a study of the following specification when viewed in the light of the accompanying drawings, in which:

FIGS. 1 and 2 are front perspective and left side elevation views, respectively, of the pyeloplasty clip of the present invention;

FIG. 3 is a sectional view illustrating the use of the clip of FIG. 1 when in a clamped condition relative to tissue of a human body;

FIG. 4 is a detailed sectional view illustrating the tissue severing operation on tissue clamped by the clip of FIG. 1, and FIG. 5 is a corresponding view illustrating the spreading of the clamped tissue when the clip is in its second separated condition;

FIG. 6 is a perspective left hand side view of a second embodiment of the pyeloplasty clip of FIG. 1, and FIG. 7 is a detailed sectional view of the fastening means of the pyeloplasty clip of FIG. 6;

FIGS. 8 and 9 are sectional views illustrating the insertion and clamping operations, respectively, of the pyeloplasty clip of FIG. 6;

FIGS. 10 and 11 illustrate the use of separation means for separating the leg portions of a clamped pyeloplasty clip;

FIG. 12 is a perspective view of a preferred form of the invention, and FIG. 13 is a modification of the clip of FIG. 12.

### DETAILED DESCRIPTION OF THE INVENTION

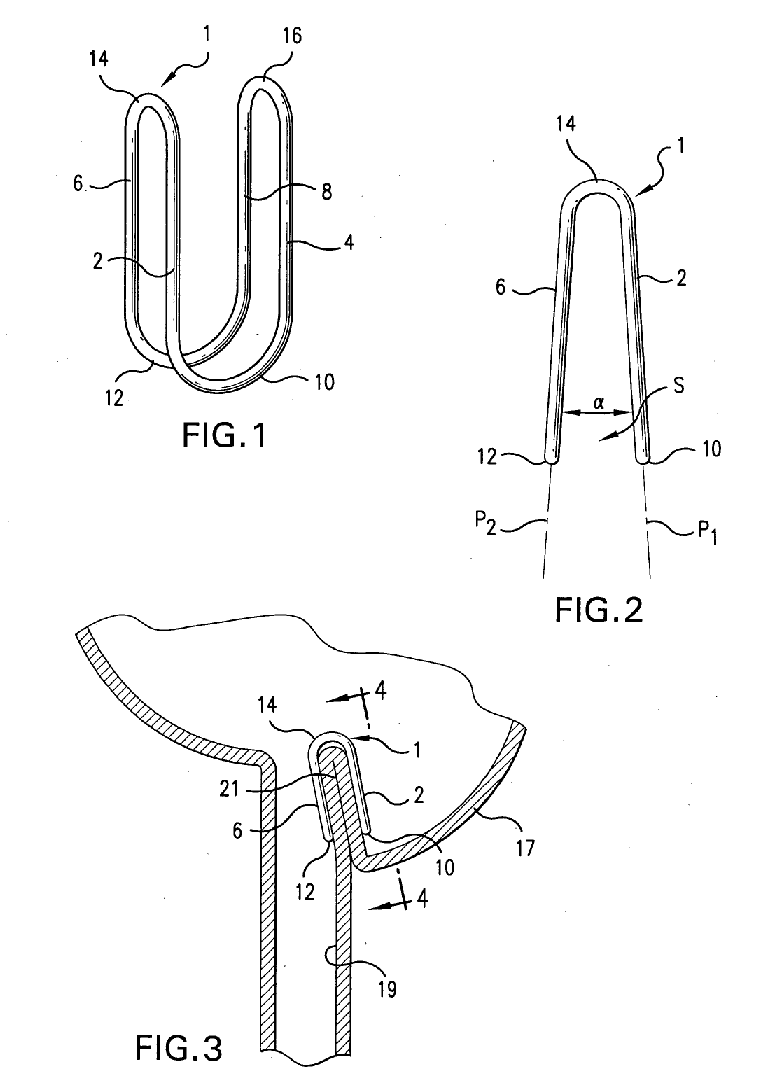

Referring first more particularly to FIGS. 1 and 2, the pyeloplasty clip 1 of the present invention includes four spaced generally parallel linear leg portions 2, 4, 6, 8. The lower ends of first and second leg portions 2 and 4 are connected by a first insertion portion 10, and the lower ends of the third and fourth leg portions 6 and 8 are connected by a second insertion portion 12. At their upper ends, the first and third leg portions 2 and 6 are connected by a first joinder portion 14, and the second and fourth leg portions 4 and 8 are connected by a second joinder portion 16. The unitary clip body is formed from a suitable metal material, such as stainless steel, shape-memory alloys, or a suitable synthetic plastic material. The linear leg portions of the clip are rigid, and the curved concave insertion and joinder portions are deformable or bendable, as will be described in greater detail below. The first and second legs 2 and 4 and the associated insertion portion 10 are contained in a common first plane P₁ (FIG. 2), and the third and fourth leg portions 6 and 8 and the associated insertion portion 12 are contained in a common second plane P₂. Preferably the planes are arranged at an acute angle α relative to the associated joinder portions 14 and 12, thereby to define a space S for receiving tissue of the human body that is to be clamped together.

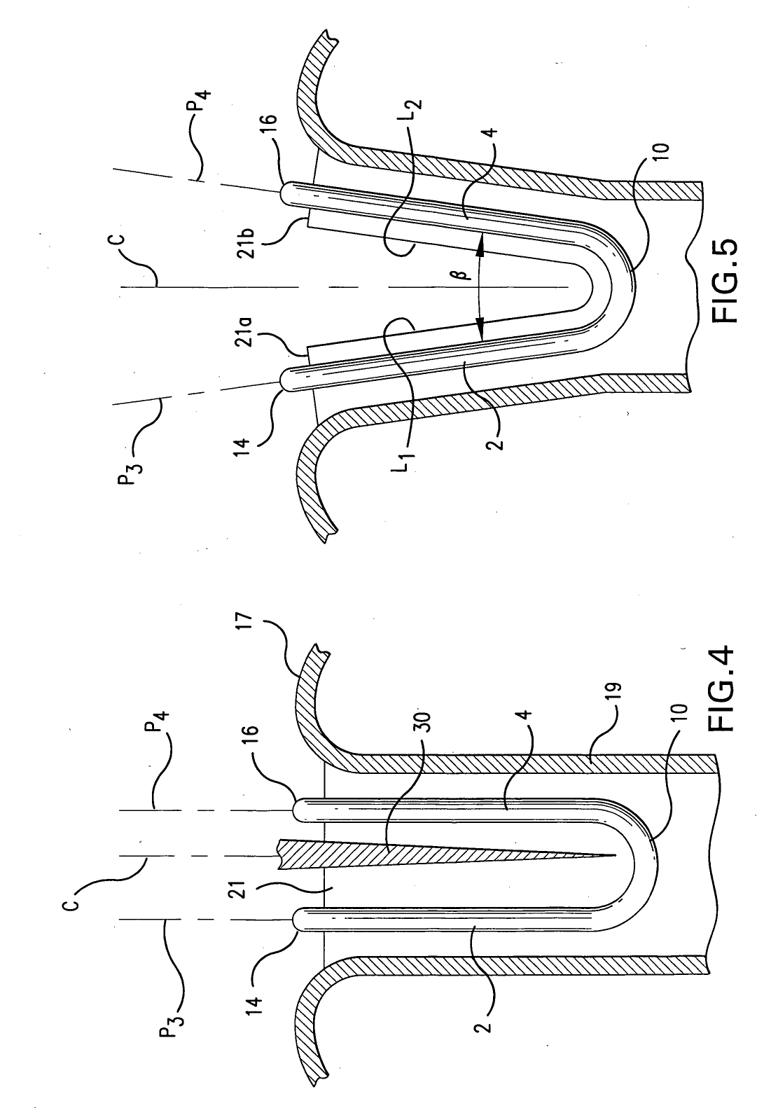

Referring now to FIGS. 3-5, when the insertion portions 10 and 12 of the pyeloplasty clip are introduced into the organ 17 and tube 19 on opposite sides of the fold of human tissue 21 that is to be clamped together, the clip is bent relative to the joinder portions 14 and 16 to cause the insertion portions 10 and 12 and the associated leg portions contained in planes P₁ and P₂, respectively, to be displaced together, and thus into a tight clamping engagement with the human tissue fold 21. The tissue fold 21 is severed by the knife means 30 (FIG. 4) along a severing line C that extends in the direction of the insertion portions 10 and 12 between a third plane P₃ containing leg and joinder portions 2, 6 and 14, and a fourth plane P₄ containing leg and joinder portions 4, 8 and 16. As shown in FIG. 5, the clamped severed tissue portions 21ₐ and 21_{b} are separated by separating the joinder portions 14 and 16 and the associate leg portions relative to the insertion portions 10 and 12 to define the acute angle β. In some cases, the pyeloplasty clip may remain in place permanently, while in other cases, the clip must be removed after the human body has healed (normally, within two to three weeks). In any event, the severed tissue portions 21ₐ and 21_{b} remain separated until the patient has healed.

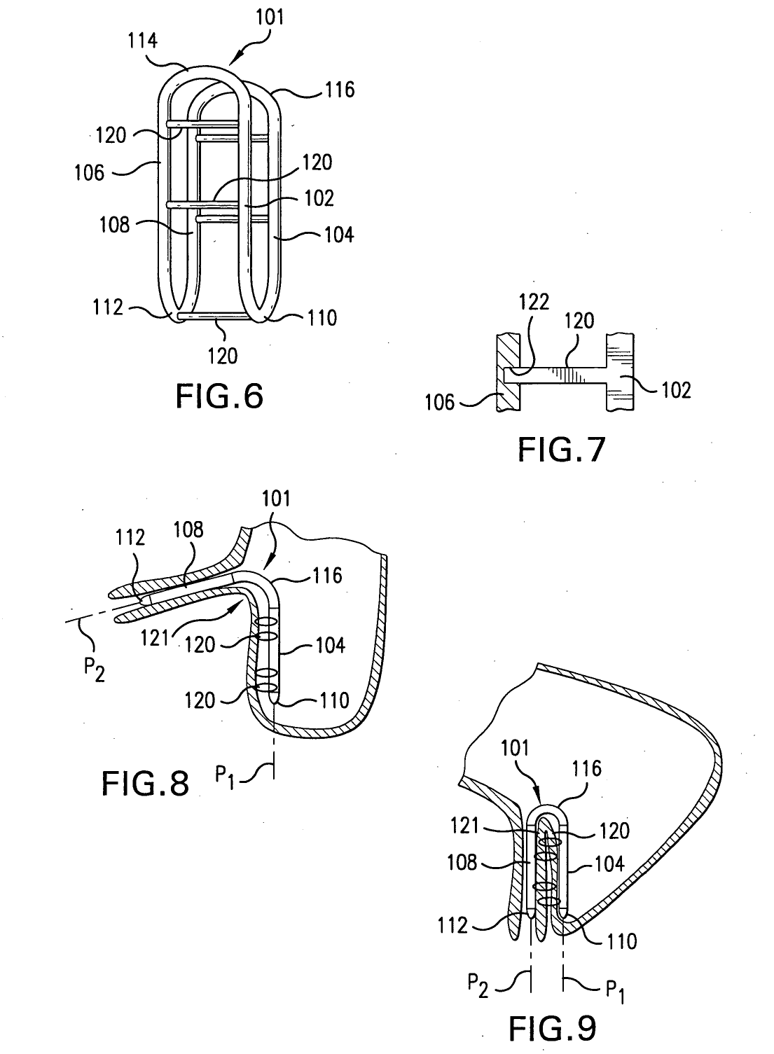

In the modification of FIGS. 6-9, fastening means are provided that are operable when the clip is in the clamped condition for fastening together the insertion and leg portions contained in plane P₁ with the insertion and leg portions contained in plane P₂. More particularly, orthogonally arranged fastening pins 120 integral with the leg portions 102 and 104 are adapted to penetrate and extend through the folded tissue portion 121 into snap fit engagement with locking openings 122 contained in the associated leg portions 106 and 108, respectively, thereby to fasten securely together the insertion and leg portions of planes P₁ and P₂ during the healing process.

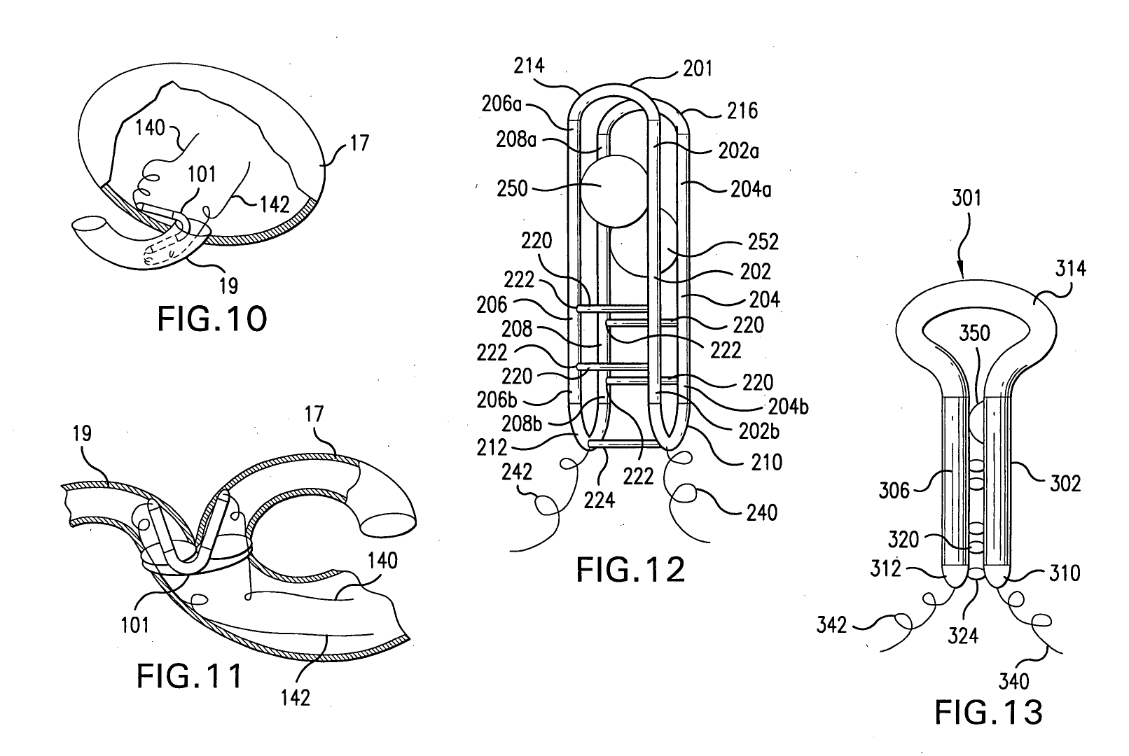

In some cases, the pyeloplasty clips (i.e., clip 1 of FIG. 3 and clip 101 of FIG. 9) may be permanently retained in the human body. Alternatively, separation means may be provided for releasing the fastening means, thereby to permit separation of the respective leg and insertions portions, and removal of the clip from the human body. As shown in FIGS. 10 and 11, the ends of the flexible string members 140 and 142 are fastened to the insertion portions of the clips. The string portions extend at their other ends from the human body via the openings used by the surgeon during the operation. After the wound has healed, the surgeon may use the strings to open the clamped leg portions for removal of the clip from the body. These string portions are provided only for those pyeloplasty clips that are to be removed.

According to another feature of the invention, pivot means may be provided for separating the clamped leg portions following the healing process. Referring now to the embodiment of FIG. 12, it will be noted that the fastening pins 220 are arranged adjacent the insertion portions 210 and 212 of the clip 201. A further fastening pin 224 extends between the insertion portions 210 and 212. Connected with the regions 202a and 204a of the leg portions 202 and 204 adjacent the joinder portions 214 and 216 are a pair of generally spherical pivot members 250 and 252 that are arranged for engagement with corresponding adjacent regions 206a and 208a of the leg portions 206 and 208, respectively. Thus, when the clip 201 is in the illustrated fastened condition, when the leg regions 202a and 206a are compressed toward the leg regions 204a and 208a, respectively, the fastening pins 220 are snapped out of engagement with the corresponding locking openings 222. The insertions portions 210 and 212 may be further separated by means of the flexible string members 240 and 242 respectively fastened thereto, thereby to permit removal of the clip from the human body.

In the modification of FIG. 13, the pivot means are defined by a pivot protrusion 350 arranged on each of the leg portions 302 and 304 for engagement with the corresponding leg portion 306 and 308, respectively. The joinder portions 314 and 316 have generally elliptical configurations, thereby to improve the pivotal operation of the legs during the separation of the fastening pins 320 from their associated locking openings.

Referring again to FIGS. 4 and 5, knife means may be provided on the leg portions 2 and 4 for severing the human tissue clamped within the clip. Thus, following the removal of the severed tissue (FIG. 4) the components in planes P₃ and P₄ (i.e., the joinder ends 14 and 16 with their attached leg portions) need only to be further separated to increase the area of fluid flow at the bifurcation.

Also, instead of forming all of the portions of the clip from the same material, some sections of the clip (such as the leg portions) could be formed from a rigid non-deformable material, while other sections (such as the insertion portions or the joinder portions) could be formed from a resilient material or a deformable material. Thus, for example, the insertion portions 14 and 16 may be resilient to bias together the components contained in planes P₁ and P₂ of FIG. 2.

While in accordance with the provisions of the Patent Statutes the preferred forms and embodiments of the invention have been illustrated and described, it will be apparent to those skilled in the art that various changes may be made without deviating from the inventive concepts set forth above.

What is claimed is:

1. A pyeloplasty clip for surgery use in clamping together two tissue portion of a human body, comprising:
a unitary clip body (l) including:
(a) first, second, third and fourth adjacent spaced generally linear leg portions (2,4,6,8);
(b) a first insertion portion (10) connecting adjacent first ends of said first and second leg portions, and a second insertion portion (12) connecting adjacent first ends of said third and fourth leg portions, said first and second leg portions and said first insertion portion being contained in a first plane (P₁), and said third and fourth leg portions and said second insertion portion being contained in a second plane (P₂); and
(c) a bendable first joinder portion (14) connecting the other ends of said first and third leg portions, and a bendable second joinder portion (16) connecting the other ends of said second and fourth leg portions, said first and third leg portions and said first joinder portion being contained in a third plane (P₃), and said second and fourth leg portions and said second joinder portion being contained in a fourth plane (P₄);
(d) said clip body normally having an expanded first condition in which the first and second insertion portions and the associated leg portions contained in said first and second planes are spaced, thereby to define a space (S) for receiving the human tissue;
(e) said clip body being operable toward a clamped second condition in which the first and second joinder portions are bent to displace said first insertion portion and the associated leg portions contained in said first plane toward said second insertion portion and the associated leg portions contained in said second plane, thereby to clamp between said insertion and leg portions the human tissue contained in said space;
(f) said clip body being operable to a displaced third condition in which said first and second insertion portions are deformed to displace said first insertion and associated leg portions contained in said third plane relative to said second insertion and associated leg portions contained in said fourth plane.

2. A pyeloplasty clip as defined in claim 1, wherein the clamped human tissue is adapted for severing along a line of severing (C) contained between said first and second joinder portions and extending in the direction of said insertion portions; and further wherein said clip body third condition is a separated condition in which said first and second joinder portions and the associated leg portions are bent outwardly to displace apart said leg and insertion portions contained in said third plane relative to said leg and insertion portions contained in said fourth plane, thereby to maintain in a separated condition the severed clamped human tissue portions.

3. A pyeloplasty clip as defined in claim 1, and further including separating means for separating the leg and insertion portions contained in one of said first and second planes from the leg and insertion portions contained in the other of said first and second planes, thereby to permit removal of the clip from the human body.

4. A pyeloplasty clip as defined in claim 1, wherein when said clip body is in its expanded first condition, said leg and insertion portions contained in said first plane are arranged at an acute angle (α) relative to the leg and insertion portions contained in said second plane.

5. A pyeloplasty clip as defined in claim 1, and further including:
(g) fastening means operable when said clip body is in said clamped second condition for fastening together the leg portions contained in said first plane with the leg portions contained in said second plane.

6. A pyeloplasty clip as defined in claim 5, wherein said joinder portions are resilient and serve to resiliently bias together the leg portions contained in said second plane (P₂) relative to the leg portions contained in said first plane (P₁).

7. A pyeloplasty clip as defined in claim 5, wherein said fastening means includes at least one fastening pin (120; 220) that extends from one of said leg portions in one of said first and second planes through the human tissue clamped therebetween into locking engagement with a corresponding locking opening (122; 222) provided in the corresponding leg portion contained in the other of said first and second planes.

8. A pyeloplasty clip as defined in claim 7, wherein said fastening means includes a further fastening pin that extends from one of said insertion portions through the human tissue into a locking opening contained in the other insertion portion.

9. A pyeloplasty clip as defined in claim 7, and further including:
(h) separating means operable when said clip body is in said clamped condition for separating said leg portions to unlock said locking pin from said locking aperture.

10. A pyeloplasty clip as defined in claim 9, wherein said separating means includes lengths of flexible members fastened to the insertion portions of said clips, respectively.

11. A pyeloplasty clip as defined in claim 7, wherein said fastening means are arranged adjacent said insertion portions; and further wherein said separating means includes pivot means (250, 252; 350) arranged between said fastening means and said joinder portions on the intermediate portion of at least one of said leg portions contained in one of said first and second planes, said pivot means being arranged for cooperation with the corresponding leg portion contained in the other of said first and second planes, whereby upon displacement together of the leg portion (202a, 206a; 204a, 208a) adjacent said joinder portion, the leg portion adjacent said insertion portion are separated.

12. A pyeloplasty clip as defined in claim 11, wherein said pivot member comprises a protrusion connected with one of said first and second legs for cooperation with the associated one of said third and fourth legs, respectively.

13. A pyeloplasty clip as defined in claim 1, wherein said first, second, third and fourth leg portions are rigid; and further wherein at least one of said leg portions includes a knife edge arranged to sever the tissue when said slip body is in the clamped condition.

14. A pyeloplasty clip as defined in claim 1, wherein each of said joining portions and said insertion portions has a curved concave configuration.

15. A pyeloplasty clip as defined in claim 14, wherein each of said joinder portions has a radius of curvature that is different than the spacing distance between the associated leg portions, respectively.

16. A pyeloplasty clip as defined in claim 15, wherein each of said joining portions has a generally semi-circular configuration.

17. A pyeloplasty clip as defined in claim 15, wherein each of said joining portions has a generally elliptical configuration.

18. A pyeloplasty clip as defined in claim 1, wherein said clip body is formed of metal, including a shape-retaining-memory metal alloy.

19. A pyeloplasty clip as defined in claim 1, wherein said clip body is formed from a synthetic plastic material.
